(19) United States
(12) Patent Application Publication
(10) Pub. No.: US 2006/0084952 A1
(43) Pub. Date: Apr. 20, 2006

(54) Device for the irradiation of the ciliary body of the eye

(76) Inventors: Ioannis G. Pallikaris, Kalessa (GR); Miltiadis K. Tsilimbaris, Heraklion (GR); Leonidas P. Naoumidis, Heraklion (GR); Harilaos S. Ginis, Heraklion (GR)

Correspondence Address: BRINKS HOFER GILSON & LIONE, P.O. BOX 10395, CHICAGO, IL, 60610, US

(21) Appl. No.: 10/934,657

(22) Filed: Sep. 3, 2004

(51) Int. Cl. A61N 5/06; A61B 18/18

(52) U.S. Cl. 606/6; 606/16; 606/13; 607/89

(57) ABSTRACT

A device is disclosed for the localized irradiation of the whole or a large part of the ciliary body of an eye. The device includes one or more optical sources and a system for the delivery of a complete or partially annular distribution of radiation to the eye.

### BACKGROUND

Increased intraocular pressure (IOP) in patients with glaucoma may induce damage to the optic nerve and subsequent loss of vision. The condition is characterized by gradual loss of optic fields, and is a major cause of blindness, accounting for approximately 10% of the total cases of blindness worldwide. While an increase of IOP alone is not sufficient for the diagnosis of the disease, it is the main indicator leading to the diagnosis of glaucoma, as well as the main cause of damage to the optic nerve.

Current methods of therapy for the treatment of glaucoma attempt to reduce the IOP. One treatment of choice for the reduction of IOP is to reduce the rate of aqueous humor production, the fluid produced by the ciliary body. Various surgical techniques, called cyclodestruction procedures, have been proposed for the partial destruction of the ciliary body and subsequent reduction of IOP. These techniques use different energy sources for the controlled destruction of the ciliary body. These techniques include trans-scleral cryotherapy, trans-scleral diathermy, and optical methods such as trans-scleral photocoagulation with an Nd:YAG laser, trans-scleral photocoagulation with a diode laser and endophotocoagulation with a diode laser, as well as photodynamic therapy of the ciliary body.

The optical methods involve devices which destroy the ciliary body spot by spot, usually by the use of an optic fiber which delivers radiation to a small section of the ciliary body. Disadvantages of these devices include the long duration of treatment required, and the reduced predictability of the degree of IOP reduction, since the damage induced by each application of radiation to the scleral region above the ciliary body is not sufficiently predictable. Also, in the case of photodynamic therapy of the ciliary body, a large dose or repeated doses of photo-sensitizers is required, so that the drug concentration in the blood is kept relatively high during the prolonged duration of the irradiation treatment.

### BRIEF SUMMARY

A device provides for localized irradiation of the ciliary body of an eye. The device includes one or more radiation sources and optical fibers for the delivery and shaping of radiation distribution. The devices allow for the simultaneous irradiation of a portion or all of the ciliary body.

The device for a localized irradiation of a ciliary body includes at least one source of radiation and a system for delivering and shaping of the radiation. A plurality of radiation conductors are utilized to simultaneously irradiate a plurality of portions of the ciliary body of the eye.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views. In the drawings:

FIG. 1 shows a perspective view of the eye to which an irradiation device is being applied.

FIG. 2 shows a side cut away view of the eye.

FIG. 3 shows a perspective view of the eye with the irradiation device being applied.

FIG. 4 shows a section of the eye, with the ciliary body, to which an optical fiber irradiation device has been applied.

FIG. 5 shows a device, the end part of which is attached to a slit lamp.

FIG. 6 shows a cross-section of the eye with the ciliary body.

FIGS. 7a and 7b show two possible configurations of the points of contact of the optical fiber ends with the sclera, according to the device shown in FIG. 4.

FIG. 8a shows a cross-section of the input end of an optical fiber bundle, where the energy from the radiation source enters the optical fiber.

FIGS. 8b and 8c show possible cross-sections of the optical fiber bundle at the output end, delivering energy to the eye.

FIG. 9 shows a device with optical elements for splitting radiation to more than one optical fiber.

FIG. 10 shows a device with optical elements for splitting radiation to more than one optical fiber after the combination to one initial fiber.

FIG. 11 shows a device that includes a number of radiation sources and a number of optical fibers.

FIG. 12 shows a cross-section of the eye, with a radiation device including an annular wave guide, which delivers controlled energy doses to the eye.

FIG. 13 shows a cross-section of the eye, with a radiation device including radiation sources placed in such a way as to direct the radiation beam perpendicular to the area of sclera, above the ciliary body.

### DETAILED DESCRIPTION

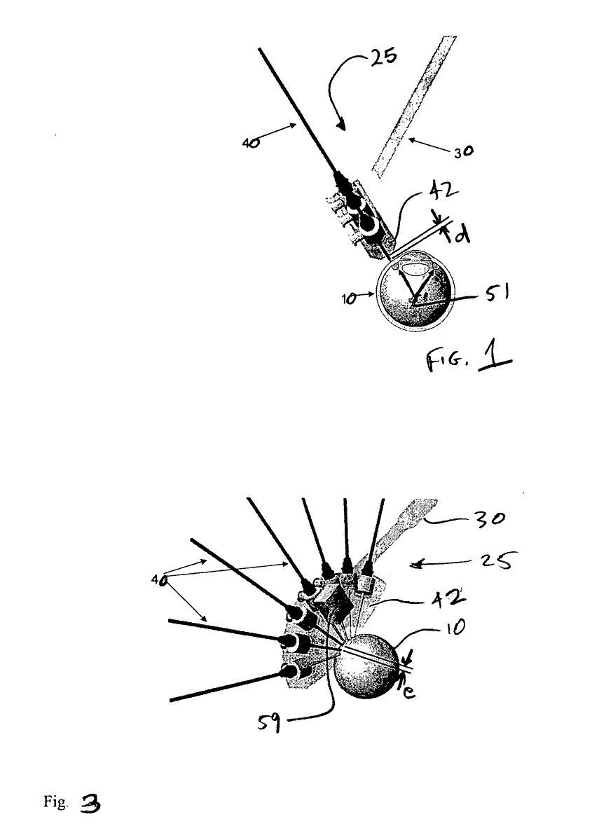

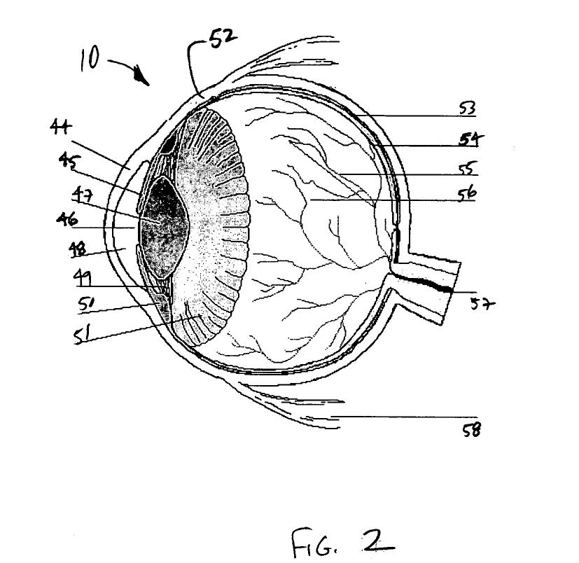

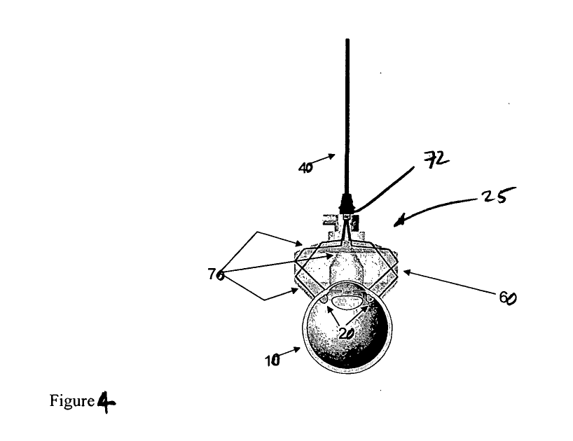

FIGS. 1 and 3 show an eye 10 and an irradiation device 25 applying radiation to the eye 10. FIG. 2 shows a side cutaway view of the eye 10. Towards a front of the eye 10, light enters a cornea 44 which overlaps an iris 45 and a lens 46. The lens 46 and the iris 45 overlap the pupil 47, and an aqueous humor 48 overlaps the lens 46. Suspensory ligaments 49 connect the pupil 47 to a ciliary muscle 50 of a ciliary body 51. The ciliary body 51 is the structure in the eye 10 that secretes the aqueous humor 48, the transparent liquid within the eye 10. The ciliary muscle 50 changes a shape of the lens 46 when the eyes 10 focus. Towards a back of the eye 10, from outside to inside, a sclera 52 overlaps a choroid 53. The choroid 53 overlaps a retina 54. Between the retina 54 and the pupil 47 arteries, veins 55 and a vitreous humor 56 are present. An optic nerve 57 connects the back of the eye 10 to the brain, and an eye muscle 58 connects to the sclera 52 of the eye 10.

Referring again to FIGS. 1 and 3, the irradiation device 25 includes a handle or hand grip 30 and at least one radiation conductor 40, such as waveguides or optical fiber, for the delivery of radiation to the eye 10. The radiation conductors 40 can be manufactured from materials such as glass, plastic and metal, which are able to transport optical and other radiation to the eye 10. Optical fibers used as radiation conductors 40 include cladding with openings in determined positions.

The radiation conductors 40 can be supported by a bracket 42 in a generally arc-like shape. In this way, the irradiation device 25 can simultaneously irradiate a defined portion of the ciliary body 51 with a determined dose of radiation. The arc-like shape of the bracket 42 allows for an annular distribution of the radiation. As used herein, annular can mean an arc-like shape, a circular shape, a semi-circular shape, and any other shape that can be formed such as a hexagon. The distribution can be solid or interspersed, such as by applying one or more points of radiation to the eye 10. The hand grip 30 attaches to the bracket 42 via interconnect 59. Alternatively, the hand grip 30 can be directly connected to the bracket 42, such that the hand grip 30 is integrally or removably attached.

The shape of the bracket 42 can be used to focus radiation emitted from the radiation conductors 40 to a common target area. In one example, the bracket 42 includes a curvature of approximately 4 to 9 mm. In another example, the arc of the bracket 42 can include a complete circle. Conducting, or free, ends of the radiation conductors 40, such as optical fibers, can be arranged in equal distances along the bracket 42 to form vertexes of a regular polygon inscribed in the circle. In another example, the conducting ends can be arranged equally distanced in the arc configuration with the arc corresponding to a central angle of approximately 45 to 180 degrees.

In one example, energy is delivered by 2 to 20 optical fibers, and more particularly 7 optical fibers, which are arranged in a general arc-like shape to simultaneously irradiate approximately one quarter of the ciliary body surface area. The hand grip 30 can be used for the perpendicular placement of the fibers on the surface of the sclera 52, and for the application of localized pressure by ends of the optical fiber on the surface of the sclera 52. The pressure can result in a determined distortion of the shape of the sclera 52. The distance d that the optical fiber protrudes from the bracket can be regulated from about 6 and about 18 mm. The clearance e (FIG. 3) between ends of the optical fibers can be set from about 0 to about 5 mm. A diameter of the optical fibers includes from about 50 to about 1000 μm. In another example, the radiation is delivered via an optical fiber bundle. One end of the bundle can include a generally circular shape and the other end can include a generally annular shape. An internal radius of the optical fiber can include a radius of curvature of approximately 4 to 9 mm, and a thickness of approximately 50 μm to 2 mm.

The distance d, the clearance e and diameter of the optical fibers are used to control application of the radiation. In one embodiment, the clearance e between two adjacent optical fibers is regulated in such a way that, after the scattering of the radiation propagated through the sclera 52, the intensity of the radiation produced on the area of the ciliary body 51 is generally homogeneous. In another embodiment, when the optical fibers are connected to a lower power source, the clearance e between two adjacent optical fibers is minimized, so that an irradiation of maximized intensity is applied to a smaller area of the ciliary body 51.

In this manner, irradiation of the ciliary body 51 of the eye 10 with irradiation device 25 can reduce the production of aqueous humor 48 and thereby reduce the intraocular pressure (IOP) of the eye 10. The irradiation device 25 allows for a greater area of the ciliary body 51 to be radiated at a time, and a total amount of time that the radiation is applied can be reduced. The reduced duration of therapy can allow for a smaller dose of photo-sensitizer to be required during photodynamic therapy of the ciliary body 51. The irradiation device 25 can also allow for an improved repeatability of the results due to the increased symmetry and dosimetry of the delivered dose of radiation.

FIG. 4 shows a section of the eye 10 and the ciliary body 51, to which the radiation from the radiation conductor 40 has been applied. The irradiation device 25 can employ a waveguide such as monolithic optical element 60. A determined amount of radiation leak takes place along the waveguide towards the eye 10. Radiation emitted by the radiation conductor 40 is reflected on the first of the conic surfaces 70 of the optical element 60. The optical element divides the radiation in sections of a generally radial propagation. The generally radially distributed radiation is subsequently directed perpendicular to the surface of the eye 10, where the ciliary body 51 is located, by way of consecutive reflections on the conic surfaces 70.

Radiation may be produced by a monolithic element 72 has two conic and one cylindrical surface. In another embodiment, the monolithic element 72 has one conic surface and one spherical, elliptic, parabolic or hyperbolic surface. In another embodiment, the monolithic element 72 has at least one conic surface and at least one additional surface of a partial cone or partially conic section. The surfaces include a common symmetry axis, and such a shape that, when the monolithic element 72 is irradiated from the symmetry axis, the element 72 produces an annular distribution of radiation according to the principles of reflection and total internal reflection. The surfaces can include a reflective coating, e.g. a metal plating. Such elements 72 can be produced by optics companies such as Melles Griot from Rochester, N.Y.

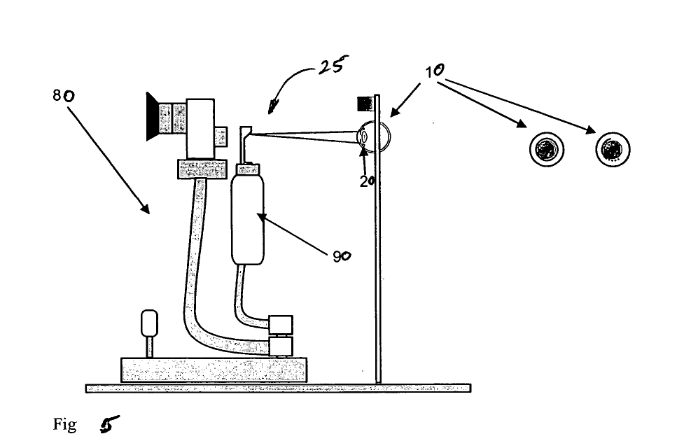

FIG. 5 shows a slit lamp 80 attached to an end part 90 to irradiate the ciliary body 51 of the eye 10 from a determined distance. The slit lamp 80 includes a microscope with a light attached that allows the user to examine the eye 10 under high magnification. This slit lamp 80 is primarily used to view the anterior structures of the eye 10 such as the cornea 44, iris 45, and lens 46. However, with special lenses, it is possible to examine the vitreous humor 56 and the back of the eye 10 as well. The slit lamp 80 includes an adjustable light beam. By changing the width of the beam, the user can gather important detail about each eye structure. The end part 90 of the slit lamp 80 can be used to deliver either a complete or partially annular distribution of radiation to the eye 10. An end part 90 propagates radiation freely to the eye 10 from a distance of approximately 5 to 30 cm from the eye 10.

The slit lamp 80 can include an eye movement detector that automatically corrects the projected position of the annular distribution of radiation to the eye 10. Eye tracking devices are typically used in refractive surgery when the laser is used to change the curvature of the cornea. To avoid errors related to movements of the eye, refractive laser systems often employ an eye tracker. Two eye trackers that can be used includes, passive, where the eye trackers sense the position of the eye and enable laser ablation only during perfect alignment, and active where the laser beam is steered appropriately to compensate for eye movements. Modern laser systems can employ active trackers such as pupil trackers that sense the pupil center by retinal reflection, and video trackers that are based on image processing techniques to locate the lateral displacement of ocular surface features, such as a limbus of the eye. Some lasers use a combination of both eye tracking systems.

The distribution of radiation may be produced by a holographic optical element. In another embodiment, the annular distribution of radiation is produced by rotating prisms for a circular scanning of a radiation beam. Scanners include optoelectric devices that are used to steer the radiation beams. Scanners are used in laser printers, light shows, medical laser systems, confocal microscopes, and some rangefinders. A common type of scanner includes a galvanometer scanner. Commercially available scanners can be used such that an annular distribution of radiation including a diameter of approximately 8 to 18 mm is delivered to the eye 10, corresponding to either a portion of all of the annulus. In an embodiment, a desired distribution of radiation is not necessarily produced by the division of the total amount of radiation to more than one beam, which are subsequently directed towards adjacent points on the surface of the eye 10. Rather, the radiation can be produced by the temporal distribution of the available power to more than one point.

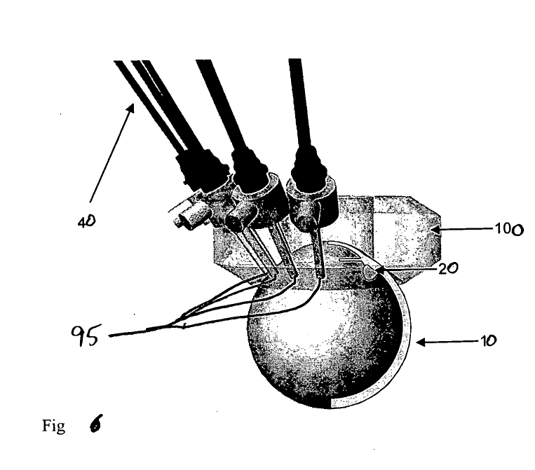

FIG. 6 shows a cross-section of the eye 10, the ciliary body 51, and a portion of the irradiation device 25 being applied to the eye. The irradiation device 25 can include an annulus or ring 100 with attached radiation conductor 40, such as optical fibers. The radiation conductors 40 are directed substantially vertically on to the surface of the eye 10, such that the points of contact of the ends 95 of the radiation conductors 40 to the eye 10, such as the sclera of the eye 10, form an arc perpendicular to the optical axis of the eye 10.

In one embodiment, the ends 95 of optical fibers protrude to apply pressure at the point of contact with the sclera 52, temporarily reducing the thickness of the sclera at that point, which can enhance the effectiveness of the applied radiation to the ciliary body 51. In another embodiment, the ring 100 supporting the optical fibers includes a suction ring for attaching the ring 100 on the surface of the eye 10. In this case, the pressure applied by the protruding optical fibers may be regulated by a vacuum pump used for engaging the ring 100 to the eye 10. The pressure in the vacuum line can be between about 100-650 mmHg lower than the atmospheric pressure.

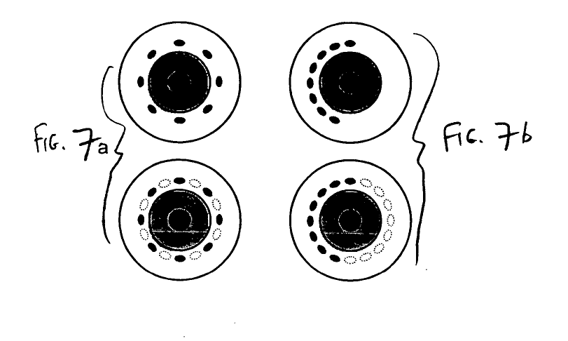

FIGS. 7a and 7b show a front view of the eye 10. FIG. 7a shows a possible configuration where the points of contact of the ends 95 with the eye 10, such as the sclera. The points shown with solid lines show the position of the ends 95 during a first application and the points shown with dotted lines show the position of the ends 95 during a second application, which can be applied consecutively to the first application. The whole ciliary body is irradiated with radiation after the first and second applications. FIG. 7b shows another configuration of the ends 95 positioned relative to the eye 10. The points shown with solid lines show the position of the ends 95 during a first application and the points shown with dotted lines show the position of the ends 95 during a second application.

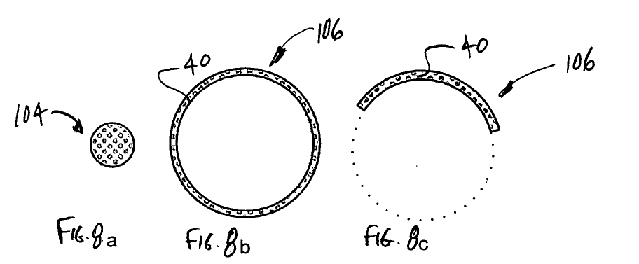

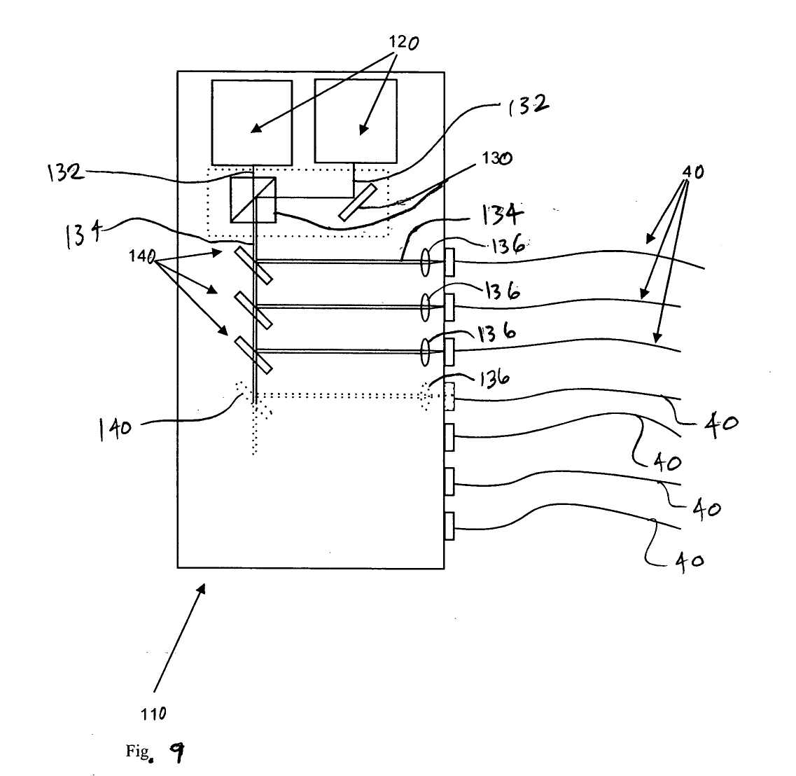

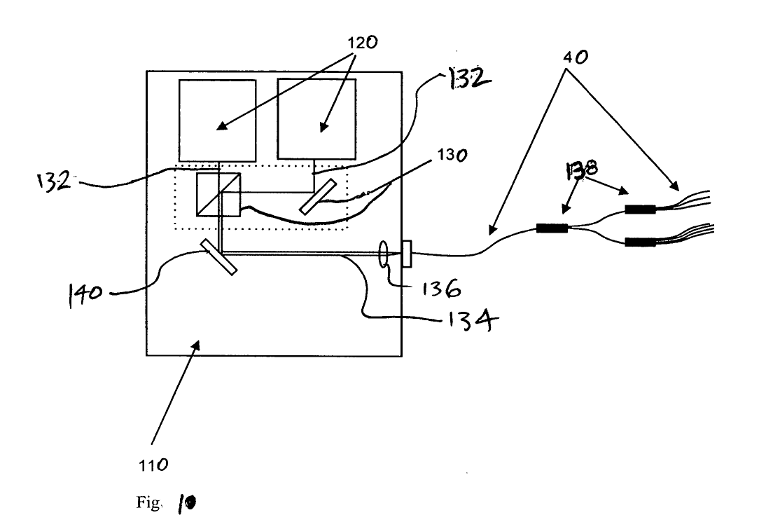

FIG. 8a shows a cross-section of the input end 104 of a radiation conductor 40, such as an optical fiber bundle. Radiation energy from the radiation source enters the optical fiber at the input 104. FIGS. 8b and 8c show possible cross-sections of the optical fiber bundle at the output end, delivering energy to the eye 10. The radiation conductors 40 can cover a complete circumference of the eye 10, as in FIG. 8b, or a partial circumference of the eye 10, as in FIG. 8c.

An annular end of the optical fiber bundle has a suction ring for attaching on the surface of the eye. Suction may be applied to the ring to maintain the fiber bundle in contact with the eye 10 and maintain the eye in an open position during the procedure.

FIG. 9 shows a radiation source device 110 that includes radiation sources 120, such as a laser, laser emitting device, including a diode laser, incandescent lamp, optical parametric oscillator, and/or electric arc lamp. The source device 110 can also include optical elements 130 for combining separate radiation beams 134 from the radiation sources 120. While two radiation sources 120 are shown, more or less than two radiation sources 120 could be used. The source device 110 can also include optical elements 140 for directing the combined radiation beam 134 towards a lens 136. The lens focuses the radiation beam to the multiple radiation conductors 40, such as optical fibers.

In one example, one radiation source 120 includes a diode laser and the other radiation source 120 includes a Nd:YAG laser. The diode laser delivers approximately 1 to 5000 mW of power. The radiation conductors 40 include optical fibers in an embodiment having a core diameter equal to 300 μm. In an embodiment, the two radiation sources 120 are combined by use of a polarizing cube beam splitter, while in another embodiment the radiation sources 120 are combined by use of dichroic mirrors. The use of two different sources can achieve simultaneously two different effects: photodynamic, at the presence of an appropriate substance, such as photo sensitizers, and pure thermal energy. The two optical configurations, beam splitter and dichroic mirrors, can combine photodynamic cylcodestruction with conventional, e.g., thermal, cyclodestruction.

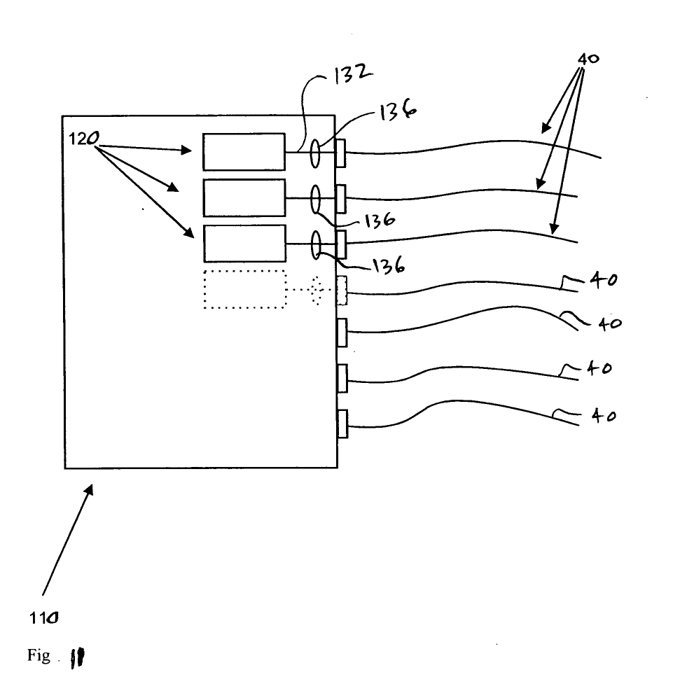

FIG. 10 shows another radiation source device 110 that includes radiation sources 120. The source device 110 includes optical elements 130 for combining separate radiation beams 134 produced by the separate radiation sources 120. The source device 110 can also include optical element 140 for directing the combined radiation beam to lens 136. The lens 136 focuses the radiation beam 136 to radiation conductors 40. Optical elements 138 can be included with the radiation conductors 40 to split the radiation from one radiation conductor 40 to multiple radiation conductors 40, such as multiple optical fibers.

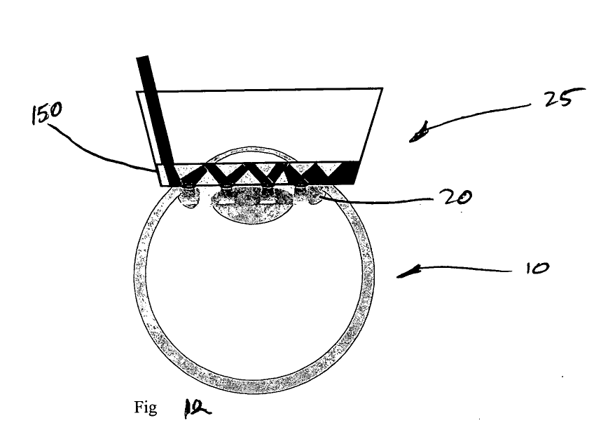

FIG. 11 shows another configuration of the source device 110. The source device 110 that includes a number of radiation sources 120 equal to the number of the employed radiation conductors 40. In this way, equal distribution of radiation can be achieved. The source device can include a diode laser device rated for power from approximately 1 to 500 mW.

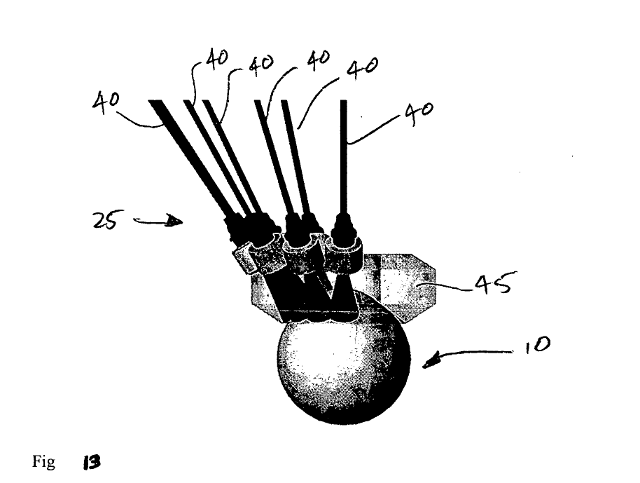

FIG. 12 shows a cross-section of the eye 10, with an irradiation device 25 including an annular configuration applied to the eye 10. The irradiation device 25 includes an annular wave guide 150, which delivers controlled energy doses to the eye 10. In one embodiment, the wave guide includes an optical fiber placed in an annular channel of the support bracket, which is placed in contact with the surface of the eye 10. The optical fiber housing has apertures on the side facing the eye 10, so that part of the propagated radiation is released towards the eye 10. The total size of the arc covered by the optical fiber inside the channel is approximately 180 degrees.

FIG. 13 shows a cross-section of the eye 10, with an irradiation device 25 of annular configuration applied to the eye 10. The irradiation device 25 includes radiation sources placed in such a way as to direct the radiation beam perpendicular to the area of sclera 52, above the ciliary body 51 of the eye 10. The optical fibers do not need to be positioned to the end of the irradiation device 25 in contact with the eye 10.

The irradiation device 25 can also include other features, such as a system for the measurement and recording of the delivered radiation dose to the eye. The irradiation device 25 can also include a system for the programmed intravenous delivery of a substance capable of amplifying the effect of radiation on the irradiated tissue. For example, upon irradiation at specified wavelengths, photosensitizers, such as phthalocyanine, verteporphin and others, undergo a chemical reaction that releases agents that can destroy adjacent tissue. This process is called photodynamic therapy in that photons are used to trigger the drug instead of directly destroying the tissue. Typically, light doses associated with photodynamic treatments are more efficient than direct laser treatments. The photodynamic therapy can use lower powered lasers, which can lower the cost of the equipment. Treatment of the tissues can be more precise since the treated tissues are specific to those that have absorbed the photosensitizers, and the low powered laser can cause less damage to surrounding tissue. Optical filters can be used to select the appropriate wavelength when polychromatic radiation is used. Differing photosensitizers can require a different characteristic wavelength, for example from green to near infrared, and more particularly red.

While the invention has been described above by reference to various embodiments, it will be understood that many changes and modifications can be made without departing from the scope of the invention. It is therefore intended that the foregoing detailed description be understood as an illustration of the presently preferred embodiments of the invention, and not as a definition of the invention. It is only the following claims, including all equivalents, which are intended to define the scope of this invention.

We claim:

1. A device for a localized irradiation of a ciliary body of an eye, the device including at least one source of radiation, the device comprising:
at least one radiation conductor to deliver radiation at a determined shape to the cilary body, where the radiation conductor is capable of simultaneously delivering the radiation to more than one portion of the ciliary body of the eye.

2. The device according to claim 1 where the radiation conductor comprises at least one of optical fibers and mirrors.

3. The device according to claim 2 where a diameter of the optical fibers comprises about 50 to about 1000 μm.

4. The device according to claim 2 where a clearance between ends of adjacent optical fibers comprises about 0 to about 5 mm.

5. The device according to claim 2 where free ends of the optical fibers are attached to a bracket having a general arc configuration, a level of the arc of the bracket being perpendicular to an optical axis of the eye, and a radius of a curvature of the arc of the bracket comprising about 4 to about 9 mm.

6. The device according to claim 5 where the arc of the bracket comprises a complete circle and the optical fiber free ends are arranged in equal distances along the circle, to form the vertexes of a regular polygon inscribed in the circle.

7. The device according to claim 5 where the free ends of the optical fibers are equally spaced along the arc of the bracket with the arc of the bracket corresponding to a central angle of about 45 to about 180 degrees.

8. The device according to claim 5 where the bracket also includes a suction ring connected with vacuum pump for attaching the bracket to the surface of the eye.

9. The device according to claim 2 where the number of optical fibers comprises from 2 to 20.

10. The device according to claim 2 where equal distribution of radiation to the optical fibers is achieved using radiation sources less in number than the number of optical fibers used, and where a distribution of radiation is achieved before the radiation enters the optical fibers.

11. The device according to claim 2 where equal distribution of radiation is achieved using radiation sources equal in number to a number of the optical fibers used.

12. The device according to claim 1 where the radiation conductor comprise an optical fiber bundle having a first end and a second end, the first end having a generally circular shape and the second end having a generally annular shape, an internal radius of curvature of the optical fiber bundle comprising about 4 to about 9 mm, and a thickness comprising about 50 μm to about 2 mm.

13. The device according to claim 12 where the end with the annular shape is placed in an arc configuration over a surface of the eye, an arc level being perpendicular to an optical axis of the eye, and an arc radius of curvature comprising about 4 to about 9 mm.

14. The device according to claim 12 where the end with the annular shape also includes a suction ring connected with a vacuum pump for attaching the end with the annular shape to the surface of the eye.

15. The device of claim 1 further comprising a radiation source device connected with the plurality of radiation conductors.

16. The device of claim 15 where radiation from the radiation source device is equally distributed to a plurality of optical fibers.

17. The device of claim 15 where radiation from the radiation source device is directed to a target point.

18. The device of claim 17 where a distance between the target point and radiation source are regulated between 6 and 18 mm.

19. The device according to claim 1 where the radiation conductor comprises a waveguide placed in contact with the eye, an arc of the waveguide being perpendicular to an optical axis of the eye, wherein a curvature of a radius of the arc comprises about 4 to about 9 mm.

20. The device according to claim 19 where the waveguide comprises an optical fiber, the optical fiber having cladding which includes openings in determined positions.

21. The device according to claim 19 where the waveguide is positioned about 5 to about 30 cm from the eye.

22. The device according to claim 19 further comprising a slit lamp, where the waveguide is attached to a slit lamp.

23. The device according to claim 19 where the waveguide delivers an annular distribution of radiation comprising a diameter of about 8 to about 18 mm.

24. The device according to claim 19 further comprising a detector for detecting eye movement, where a projected position of distributed radiation is automatically adjusted in accordance with the eye movement.

25. The device according to claim 19 where the waveguide comprises at least one rotating prism to produce an annular distribution of radiation.

26. The device according to claim 19 where the waveguide comprises holographic elements to produce an annular distribution of radiation.

27. The device according to claim 19 where the waveguide comprises a galvanometric optical scanner to produce an annular distribution of radiation.

28. The device according to claim 1 where the sources of radiation comprises at least one of a laser, a laser emitting device, an incandescent lamp, and an electric arc lamp.

29. The device according to claim 1 where the source of radiation comprises a diode laser having a power of about 1 to about 5000 mW.

30. The device according to claim 1 where the source of radiation comprises an optical parametric oscillator.

31. The device according to claim 1 where the source of radiation comprises a first source of radiation and a second source of radiation, where the first source of radiation comprises a diode laser and the second source of radiation comprises an Nd:YAG laser.

32. The device according to claim 1 where the source of radiation comprises multiple diode laser emitting devices including a power of about 1 to about 500 mW.

33. Thedevice according to claim 1 further comprising a delivery device to conduct an intravenous delivery of a substance capable of amplifying the effect of radiation on the ciliary body of the eye.

34. The device according to claim 1 further comprising a measuring device to measure a delivered dose of radiation to the eye.

35. The device according to claim 1 where a geometric configuration of the radiation conductor enables pressure to be applied on the surface of the sclera, resulting in the distortion of a shape of the sclera.

36. The device according to claim 1 where the radiation conductor comprises at least one first surface having conic shape and at least one second surface having a partially conic shape, the first surface and the second surface have a common symmetry axis and a shape such that when the radiation conductor is irradiated from a symmetry axis, an annular distribution of radiation is produced.
